(19) United States
(12) Patent Application Publication
(10) Pub. No.: US 2005/0085640 A1
(43) Pub. Date: Apr. 21, 2005

(54) Novel crystalline forms of gatifloxacin

(75) Inventors: Reddy Bandi Parthasaradhi, Hyderabad (IN); Reddy Kura Rathnakar, Hyderabad (IN); Reddy Rapolu Raji, Hyderabad (IN); Reddy Dasari Muralidhara, Hyderabad (IN); Reddy Meghi Ravikanth, Hyderabad (IN)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: HETERO DRUGS LIMITED, Hyderabad, Andhrajpradesh (IN)

(21) Appl. No.: 10/510,172

(22) PCT Filed: Apr. 2, 2003
(86) PCT No.: PCT/IN03/00135

(51) Int. Cl. A61K 31/496; C07D 43/02

(52) U.S. Cl. 544/363; 514/253.08

(57) ABSTRACT

The present invention relates to novel crystalline forms of gatifloxacin, to processes for their preparation and to pharmaceutical compositions containing them.

### FIELD OF THE INVENTION

The present invention relates to novel crystalline forms of gatifloxacin, to processes for their preparation and to pharmaceutical compositions containing them.

### BACKGROUND OF THE INVENTION

Gatifloxacin of formula (1): 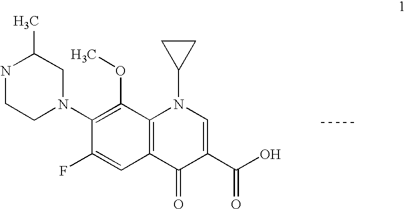
or 1-Cyclopropyl-6-fluoro-1,4-dihydro-8-methoxy-7-(3-methyl-1-piperazinyl)-4-oxo-3-quinolinecarboxylic acid is an antibacterial agent and its therapeutic uses are disclosed in U.S. Pat. No. 4,980,470.

Example 3 and 14 of U.S. Pat. No. 4,980,470 described monohydrate of gatifloxacin. A crystalline form of sesquihydrate of gatifloxacin is disclosed in U.S. Pat. No. 5,880,283. Various crystalline forms of gatifloxacin hydrates are mentioned in U.S. Pat. No. 6,413,969.

We have discovered five stable novel crystalline forms of gatifloxacin and these forms are found to be suitable for pharmaceutical preparations.

The object of the present invention is to provide stable novel crystalline forms of gatifloxacin, processes for preparing these forms and pharmaceutical compositions containing them.

### DETAILED DESCRIPTION OF THE INVENTION

In accordance with the present invention, there is provided a novel crystalline form of gatifloxacin sesquihydrate, designated as Form H1, characterized by an x-ray powder diffraction pattern having peaks expressed as 2θ at about 9.2, 10.5, 12.9, 18.4, 18.9, 19.9, 21.2, 21.7 and 24.0 degrees. FIG. 1 shows typical Form H1 x-ray powder diffraction pattern.

Gatifloxacin sesquihydrate Form H1 is prepared by crystallizing gatifloxacin sesquihydrate Form H1 from a solution comprising gatifloxacin, a chlorinated solvent and water. The suitable chlorinated solvents are ethylene dichloride, chloroform, carbon tetrachloride and methylene dichloride. A mixture of chlorinated solvents is also part of the invention. The water content in the solution should be at least 1.5 mole per mole of gatifloxacin. There is no upper limit for water content so long as gatifloxacin sesquihydrate Form H1 can be crystallized from the solution. A hydrated form of gatifloxacin or gatifloxacin prepared by a known method may be used in the process. Preferably, gatifloxacin sesquihydrate Form H1 is crystallized at about 20° C. to 25° C. from the solution.

In accordance with the present invention, there is provided a novel crystalline form of gatifloxacin, designated as Form H2, characterized by an x-ray powder diffraction pattern having peaks expressed as 2° at about 5.9, 7.8, 13.7, 14.1, 15.9, 19.7 and 21.1 degrees. FIG. 2 shows typical Form H2 x-ray powder diffraction pattern.

In accordance with the present invention, a process is provided for preparation of gatifloxacin Form H2. In this process, gatifloxacin is mixed with an ester solvent at a higher temperature, preferably at about 70° C. to 80° C., cooling the contents rapidly to about 20° C. to 25° C. and filtering gatifloxacin Form H2 from the contents at about 20° C. to 25° C. The suitable ester solvents are ethyl acetate, methyl acetate, isopropyl acetate, tert-butyl acetate, ethyl formate and methyl formate. A mixture of the ester solvents may also be used. A hydrated form of gatifloxacin or gatifloxacin prepared by a known method may be used in the process. The mixture of gatifloxacin and the ester solvent is preferably maintained at 70° C. to 80° C. for about 30 minutes, cooled to at about 20° C. to 25° C. in about 1 hour and then maintained for about 12 hours at about 20° C. to 25° C.

In accordance with the present invention, there is provided a novel crystalline form of gatifloxacin, designated as Form H3, characterized by an x-ray powder diffraction pattern having peaks expressed as 2θ at about 7.8, 10.2, 12.9, 13.6, 14.1, 19.7, 20.5, 23.8, 25.9 and 28.6 degrees. FIG. 3 shows typical Form H3 x-ray powder diffraction pattern.

In accordance with the present invention, a process is provided for preparation of gatifloxacin Form H3. In this process, gatifloxacin is mixed with an ester solvent at a higher temperature, preferably at about 70° C. to 80° C., cooling the contents slowly to about 20° C. to 25° C. and filtering gatifloxacin Form H3 from the contents at about 20° C. to 25° C. The suitable ester solvents are ethyl acetate, methyl acetate, isopropyl acetate, tert-butyl acetate, ethyl formate and methyl formate. A mixture of the ester solvents may also be used. A hydrated form of gatifloxacin or gatifloxacin prepared by a known method may be used in the process. The mixture of gatifloxacin and the ester solvent is preferably maintained at 70° C. to 80° C. for about 30 minutes, cooled to at about 20° C. to 25° C. in about 4 to 6 hours and then maintained for about 12 hours at about 20° C. to 25° C.

In accordance with the present invention, there is provided a novel crystalline form of gatifloxacin sesquihydrate, designated as Form H4, characterized by an x-ray powder diffraction pattern having peaks expressed as 2θ at about 6.3, 7.8, 9.2, 9.8, 10.6, 12.6, 12.9, 13.5, 14.4, 18.4, 19.8, 20.0, 20.9, 24.4, 25.4, 25.9 and 27.9 degrees. FIG. 4 shows typical Form H4 x-ray powder diffraction pattern.

Gatifloxacin sesquihydrate Form H4 is prepared by crystallizing gatifloxacin sesquihydrate Form H4 from a solution comprising gatifloxacin, a suitable quantity of 1,4-dioxane and water. The quantity of the 1,4-dioxane is above 20 ml, preferably 20 to 40 ml per gm of gatifloxacin. The water content in the solution should be at least 1.5 mole per mole of gatifloxacin. There is no upper limit for water content so long as gatifloxacin sesquihydrate Form H4 can be crystallized from the solution. A hydrated form of gatifloxacin or gatifloxacin prepared by a known method may be used in the process.

In accordance with the present invention, there is provided a novel crystalline form of gatifloxacin sesquihydrate, designated as Form H5, characterized by an x-ray powder diffraction pattern having peaks expressed as 2θ at about 8.2, 13.5, 13.9, 16.5, 17.0, 17.9, 19.9, 21.0, 23.3 and 24.8 degrees. FIG. 5 shows typical Form H5 x-ray powder diffraction pattern.

Gatifloxacin sesquihydrate Form H5 is prepared by crystallizing gatifloxacin sesquihydrate Form H5 from a solution comprising gatifloxacin, a suitable quantity of 1,4-dioxane and water. The quantity of the 1,4-dioxane is below 20 ml, preferably 8 to 15 ml per gm of gatifloxacin. A hydrated form of gatifloxacin or gatifloxacin prepared by a known method may be used in the process. The water content in the solution should be at least 1.5 mole per mole of gatifloxacin. There is no upper limit for water content so long as gatifloxacin sesquihydrate Form H5 can be crystallized from the solution.

In accordance with the present invention, there is provided a pharmaceutical composition comprising any of the crystalline forms, Form H1 to H5, of gatifloxacin and a pharmaceutically acceptable carrier.

### BRIEF DESCRIPTION OF THE DRAWINGS

FIG. 1 is a x-ray powder diffraction spectrum of gatifloxacin sesquihydrate Form H1.

FIG. 2 is a x-ray powder diffraction spectrum of gatifloxacin Form H2.

FIG. 3 is a x-ray powder diffraction spectrum of gatifloxacin Form H3.

FIG. 4 is a x-ray powder diffraction spectrum of gatifloxacin sesquihydrate Form H4.

FIG. 5 is a x-ray powder diffraction spectrum of gatifloxacin sesquihydrate Form H5.

x-Ray powder diffraction spectrum was measured on a Siemens D5000 x-ray powder diffractometer having a copper-Kα radiation.

The following examples further illustrate the invention.

### EXAMPLE 1

Gatifloxacin hemihydrate (1 gm) (obtained by the process described in example-3 of U.S. Pat. No. 4,980,470) is mixed with ethylene dichloride (20 ml, water content 0.3% w/w), heated to 45° C. and maintained at this temperature for 15 minutes. The clear solution formed is cooled to 25° C. and maintained at 25° C. for 12 hours. The separated crystals are filtered to give 0.7 gm of gatifloxacin sesquihydrate Form H1.

### EXAMPLE 2

Gatifloxacin (1 gm) is mixed with methylene dichloride (50 ml, water content 0.35% w/w), heated to 45° C. and maintained at this temperature for 15 minutes. The solution formed is cooled to 25° C. and maintained at 25° C. for 10 hours. The separated crystals are filtered to give 0.6 gm of gatifloxacin sesquihydrate Form H1.

### EXAMPLE 3

Gatifloxacin monohydrate (1 gm) is mixed with ethyl acetate (35 ml), heated to 75° C. and maintained at this temperature for 15 minutes. The solution is cooled rapidly to 25° C. in 1 hour and maintained for about 12 hours at 25° C. The separated crystals are filtered to give 0.5 gm of gatifloxacin Form H2.

### EXAMPLE 4

Example 3 is repeated using gatifloxacin sesquihydrate Form H1 for gatifloxacin monohydrate to give gatifloxacin Form H2.

### EXAMPLE 5

Gatifloxacin monohydrate (10 gm) is mixed with ethyl acetate (350 ml), heated to reflux and maintained at this temperature for 15 minutes. The solution is cooled slowly to 25° C. in 5 hours and maintained for about 10 hours at 25° C. The separated crystals are filtered to give 6.0 gm of gatifloxacin Form H3.

### EXAMPLE 6

Example 5 is repeated using gatifloxacin Form H2 for gatifloxacin monohydrate to give gatifloxacin Form H3.

### EXAMPLE 7

Gatifloxacin (1.0 gm) is mixed with 1,4-dioxane (30 ml, water content 0.4% w/w), refluxed for 10 minutes. The solution obtained is cooled to 25° C. for about 12 hours. The separated crystals are filtered to give 0.8 gm of gatifloxacin sesquihydrate Form H4.

### EXAMPLE 8

Example 7 is repeated using gatifloxacin Form H3 for gatifloxacin to give gatifloxacin sesquihydrate Form H4.

### EXAMPLE 9

Gatifloxacin (10 gm) is mixed with 1,4-dioxane (100 ml, water content 0.4% w/w), refluxed for 15 minutes. The solution obtained is cooled to 25° C. for about 10 hours. The separated crystals are filtered to give 9.2 gm of gatifloxacin sesquihydrate Form H5.

### EXAMPLE 10

Example 9 is repeated using gatifloxacin sesquihydrate Form H1 for gatifloxacin to give gatifloxacin sesquihydrate Form H5.

1. A crystalline gatifloxacin sesquihydrate Form H1, characterized by an x-ray powder diffraction pattern having peaks expressed as 2θ at about 9.2, 10.5, 12.9, 18.4, 18.9, 19.9, 21.2, 21.7 and 24.0 degrees.

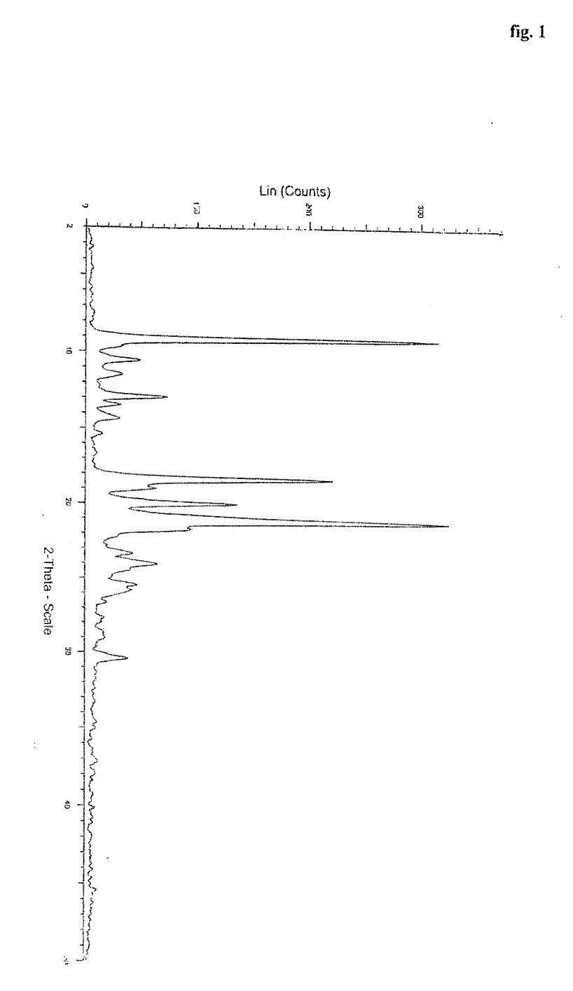

2. The crystalline gatifloxacin sesquihydrate Form H1 as defined in claim 1, further characterized by an x-ray powder diffraction pattern as in FIG. 1.

3. The process for preparation of gatifloxacin sesquihydrate Form H1 as defined in claim 1, which comprises crystallizing gatifloxacin sesquihydrate Form H1 from a solution comprising gatifloxacin, a chlorinated solvent and water;
wherein the chlorinated solvent is selected from the group consisting of ethylene dichloride, chloroform, carbon tetrachloride and methylene dichloride.

4. The process according to claim 3, wherein the chlorinated solvent is ethylene dichloride.

5. The process according to claim 3, wherein gatifloxacin is a hydrate of gatifloxacin.

6. The crystalline gatifloxacin Form H2, characterized by an x-ray powder diffraction pattern having peaks expressed as 2θ at about 5.9, 7.8, 13.7, 14.1, 15.9, 19.7 and 21.1 degrees.

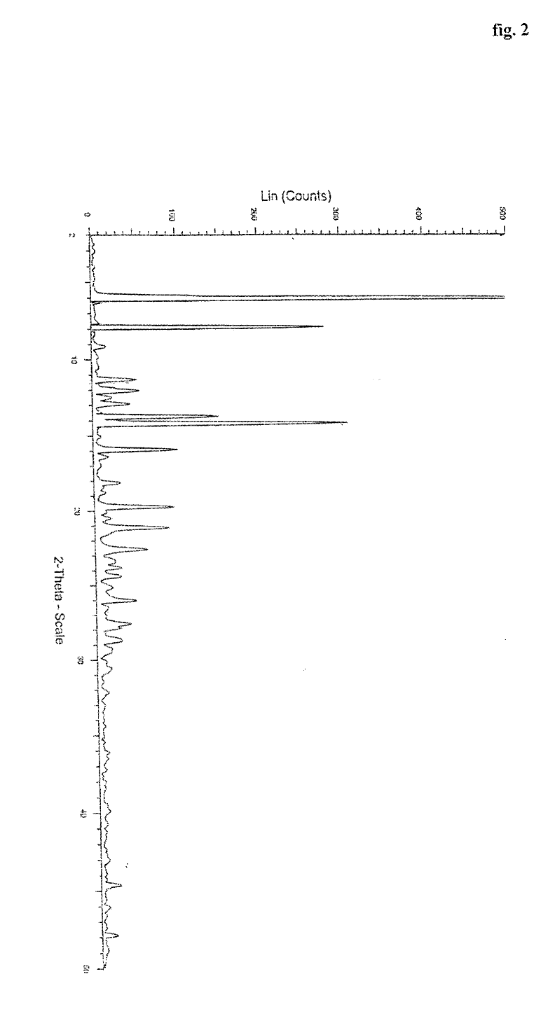

7. The crystalline gatifloxacin Form H2 as defined in claim 6, further characterized by an x-ray powder diffraction pattern as in FIG. 2.

8. The process for preparation of gatifloxacin Form H2 as defined in claim 6, which comprises the steps of:
a) mixing gatifloxacin and an ester solvent;
b) heating to about 70° C. to 80° C.;
c) cooling rapidly to about 20° C. to 25° C.; and
d) filtering the solid separated;
wherein the ester solvent is selected from the group consisting of ethyl acetate, methyl acetate, isopropyl acetate, tert-butyl acetate, ethyl formate and methyl formate.

9. The process according to claim 8, wherein the gatifloxacin used is gatifloxacin sesquihydrate Form H1.

10. The process according to claim 8, wherein the ester solvent is ethyl acetate.

11. The process according to claim 8, wherein the contents are cooled to about 20° C. to 25° C. in 1 hour.

12. The process according to claim 3, wherein gatifloxacin used is gatifloxacin Form H2 of claim 6.

13. A crystalline gatifloxacin Form H3, characterized by an x-ray powder diffraction pattern having peaks expressed as 2θ at about 7.8, 10.2, 12.9, 13.6, 14.1, 19.7, 20.5, 23.8, 25.9 and 28.6 degrees.

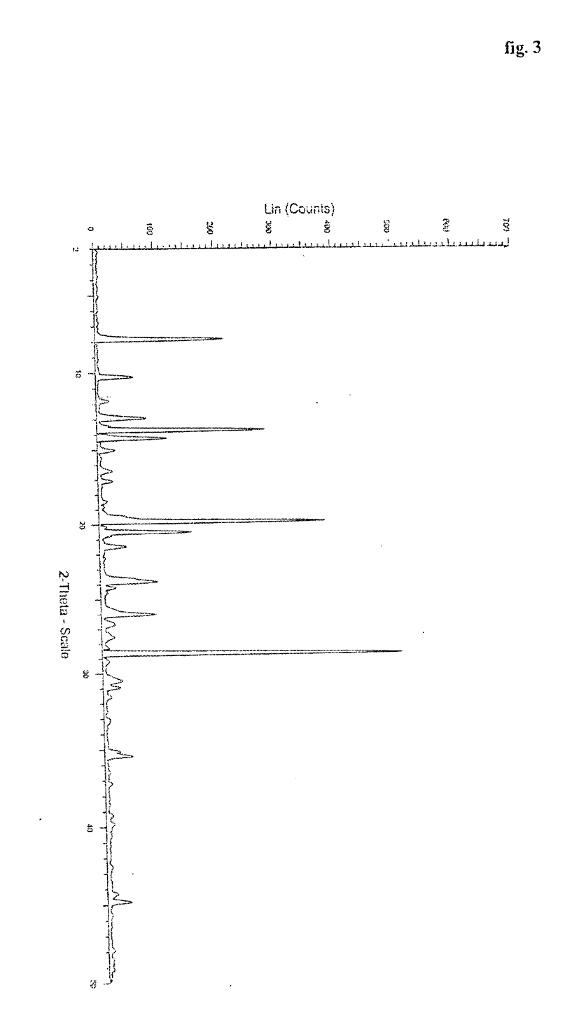

14. The crystalline gatifloxacin Form H3 as defined in claim 13, further characterized by an x-ray powder diffraction pattern as in FIG. 3.

15. The process for preparation of gatifloxacin Form H3 as defined in claim 13, which comprises the steps of:
a) mixing gatifloxacin and an ester solvent;
b) heating to about 70° C. to 80° C.;
c) cooling slowly to about 20° C. to 25° C.; and
d) filtering the solid separated;
wherein the ester solvent is selected from the group consisting of ethyl acetate, methyl acetate, isopropyl acetate, tert-butyl acetate, ethyl formate and methyl formate.

16. The process according to claim 15, wherein gatifloxacin used is hydrate of gatifloxacin.

17. The process according to claim 15, wherein gatifloxacin used is gatifloxacin Form H2.

18. The process according to claim 15, wherein the contents are cooled to about 20° C. to 25° C. in 4 to 6 hours.

19. The process according to claim 3, wherein gatifloxacin used is gatifloxacin Form H3 of claim 13.

20. The process according to claim 8, wherein gatifloxacin used is gatifloxacin Form H3 of claim 13.

21. A crystalline gatifloxacin sesquihydrate Form H4, characterized by an x-ray powder diffraction pattern having peaks expressed as 2θ at about 6.3, 7.8, 9.2, 9.8, 10.6, 12.6, 12.9, 13.5, 14.4, 18.4, 19.8, 20.0, 20.9, 24.4, 25.4, 25.9 and 27.9 degrees.

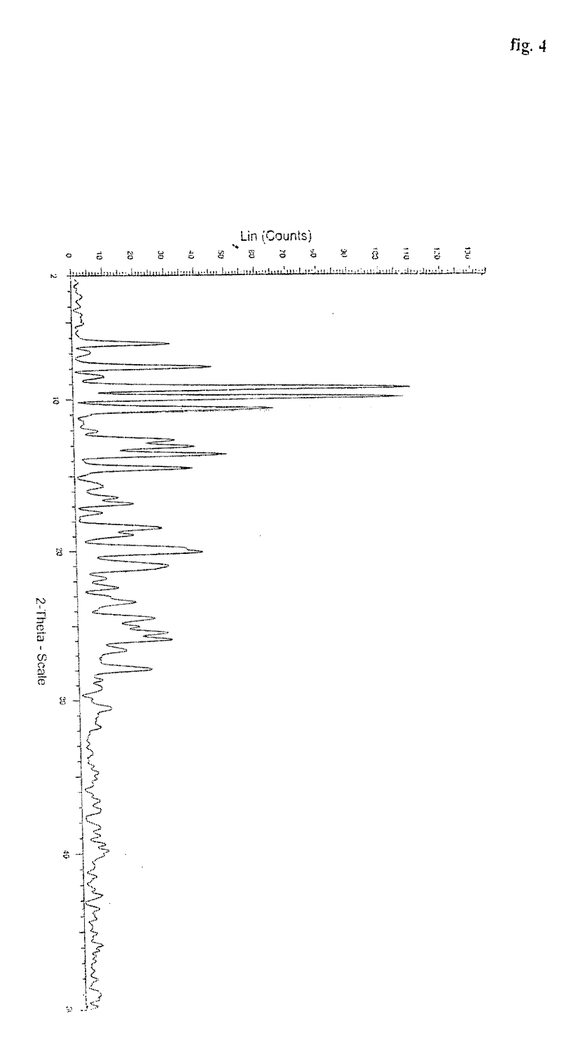

22. The crystalline gatifloxacin sesquihydrate Form H4 as defined in claim 21, further characterized by an x-ray powder diffraction pattern as in FIG. 4.

23. The process for preparation of gatifloxacin sesquihydrate Form H4 as defined in claim 21, which comprises crystallizing gatifloxacin sesquihydrate Form H4 from the solution comprising gatifloxacin, a suitable quantity of 1,4-dioxane and water; wherein the quantity of 1,4-dioxane is above 20 ml per gm of gatifloxacin.

24. The process according to claim 23, wherein the quantity of 1,4-dioxane is 20 to 40 ml per gm of gatifloxacin.

25. The process according to claim 23, wherein the gatifloxacin is a hydrate of gatifloxacin.

26. A crystalline gatifloxacin sesquihydrate Form H5, characterized by an x-ray powder diffraction pattern having peaks expressed as 2θ at about 8.2, 13.5, 13.9, 16.5, 17.0, 17.9, 19.9, 21.0, 23.3 and 24.8 degrees.

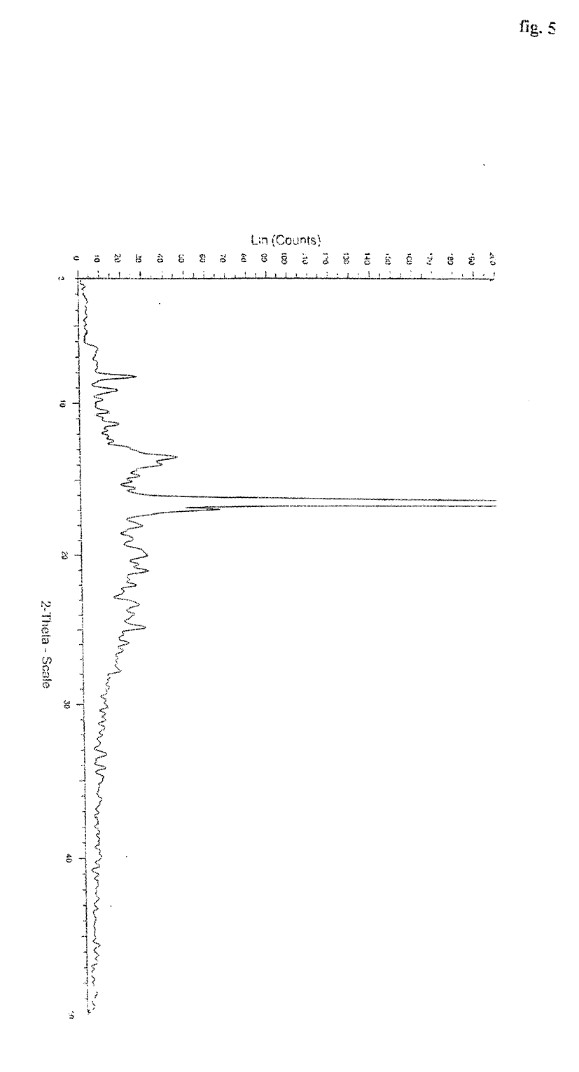

27. The crystalline gatifloxacin sesquihydrate Form H5 as defined in claim 26 further characterized by an x-ray powder diffraction pattern as in FIG. 5.

28. The process for preparation of gatifloxacin sesquihydrate Form H5 as defined in claim 26, which comprises crystallizing gatifloxacin sesquihydrate Form H5 from the solution comprising gatifloxacin, a suitable quantity of 1,4-dioxane and water; wherein the quantity of 1,4-dioxane is equal to or below 20 ml per gm of gatifloxacin.

29. The process according to claim 28, wherein the quantity of 1,4-dioxane is 8 to 15 ml per gm of gatifloxacin.

30. The process according to claim 28, wherein the gatifloxacin is a hydrate of gatifloxacin.

31. The pharmaceutical composition comprising a crystalline form of gatifloxacin and a pharmaceutically acceptable carrier; wherein the crystalline form is selected from the group consisting of Form H1 of claim 1, Form H2 of claim 6, Form H3 of claim 13, Form H4 of claim 21 and Form H5 of claim 26.

32. The pharmaceutical composition of claim 31 wherein the crystalline form is gatifloxacin sesquihydrate Form H1 of claim 1.

33. The pharmaceutical composition as defined in claim 31, wherein the crystalline form is gatifloxacin Form H2 of claim 6.

34. The pharmaceutical composition as defined in claim 31, wherein the crystalline form is gatifloxacin Form H3 of claim 13.

35. The pharmaceutical composition as defined in claim 31 wherein the crystalline form is gatifloxacin sesquihydrate Form H4 of claim 21.

36. The pharmaceutical composition as defined in claim 30, wherein the crystalline form is gatifloxacin sesquihydrate Form H5 of claim 26.
